(19) United States
(12) Patent Application Publication
(10) Pub. No.: US 2025/0229956 A1
(43) Pub. Date: Jul. 17, 2025

(54) CONTAINER FOR METERED DISPENSING OF A FLUID

(71) Applicant: PACKSYS GMBH, Feldafing/OT Wieling (DE)

(72) Inventors: Sybille BURKHARDT, Lauchheim (DE); Josef THIERMEYER, Unterammergau (DE); Veronika LÖSSL, Rosenheim (DE); Markus FINGER, Starnberg (DE)

(21) Appl. No.: 18/869,185

(22) PCT Filed: May 25, 2023
(86) PCT No.: PCT/EP2023/064046

§ 371 Date: Nov. 25, 2024

(30) Foreign Application Priority Data
May 27, 2022 (EP) 22175769.3

(51) Int. Cl. B65D 47/18; A61F 9/00; B65D 1/02

(52) U.S. Cl. CPC B65D 47/18; A61F 9/0008; B65D 1/0261

(57) ABSTRACT

The invention relates to a container for metered dispensing of a sterile fluid, the container comprising a substantially rotationally symmetrical storage body (12) made of plastics material, having a longitudinal axis (O) and a radial direction (R) extending perpendicularly to the longitudinal axis, wherein the storage body (12) comprises a substantially cylindrical or conical casing portion (14) having a first wall thickness (S1), a metering portion (16), a bottom portion (18) and a container neck portion (20), characterized in that the metering portion (16) is arranged between the casing portion (14) and the bottom portion (18), and is configured as a folding bellows having at least one radially inward fold having pivot points (36, 38, 40); wherein the wall thickness (S2) of the metering portion (16) is reduced, at least in the region of the joints (36,38, 40), by 25% to 60% with respect to the first wall thickness S1.

### FIELD OF THE INVENTION

The invention relates to a container for the metered dispensing of a sterile fluid.

### BACKGROUND OF THE INVENTION

Sterile fluids, such as eye drops, are either supplied in individual doses or dispensed from containers that contain the quantity of sterile fluid for multiple applications. Eye drops are dripped into the conjunctival sac or onto the cornea by the user and must be sterile to prevent eye infections.

If sterile fluids are to be dispensed in precisely prescribed doses in the form of a certain number of drops, containers have been proposed in the prior art with which a defined partial quantity can be dispensed from the container. To this end, the storage container is provided with an elastic side wall, for example, which can be compressed by the user during use in order to be able to dispense one or more individual drops. When dispensing eye drops, the container is usually placed upside down so that the dispensed drops essentially reach the user's eye from above.

In the prior art, containers have also been proposed whose bottom wall can assume two defined states. By applying pressure to the bottom wall, a container bottom that is curved towards the outside of the container in the first position can be moved to a second position in which the bottom is curved towards the inner volume of the container. During the movement from the first position to the second position, a defined volume is displaced inside the container, allowing a defined quantity of fluid to be dispensed from a dispensing opening.

Since the function of such a bottom, which can assume various geometric states, is not intuitively recognizable for a user, there is a need for a container for the metered dispensing of a sterile fluid, the operation of which is already recognizable and deducible for an average user from its shape. At the same time, the container must be easy to manufacture since such containers are mass-produced.

### DESCRIPTION OF THE INVENTION

This object is solved by a container for the metered dispensing of a sterile fluid having the features of claim 1. Preferred embodiments are apparent from the other claims.

A container for the metered dispensing of a sterile fluid, as according to the invention, comprises a substantially rotationally symmetrical storage body made of plastics material, having a longitudinal axis and a radial direction extending perpendicularly to the longitudinal axis, wherein the storage body comprises a substantially cylindrical or conical casing portion having a first wall thickness, a metering portion, a bottom portion and a container neck portion. The container is characterized in that the metering portion is arranged between the casing portion and the bottom portion, and is configured as a folding bellows having at least one radially inward fold having joints, wherein the wall thickness of the metering portion is reduced, at least in the region of the joints, by 25% to 60% with respect to the first wall thickness.

The container according to the invention thus comprises a metering portion arranged between the casing portion and the bottom portion of the container, which is arranged so as to be clearly recognizable in the region of the casing area of the container. This metering portion is configured as a folding bellows, and thus the folding radially inward is easily visible for a user. Therefore, a user also intuitively recognizes how to use the container according to the invention. The use is enabled in that the wall thickness of the metering portion is provided with a reduced wall thickness, at least in the region of the joints of the at least one fold, wherein the wall thickness is reduced, at least in the region of the joints, by 25% to 60% with respect to the wall thickness of the substantially cylindrical or conical casing portion. Due to the reduced wall thickness of the joints, the integrally manufactured container can be folded in the region of the fold by reducing the height of the container when pressure is applied to the bottom portion since the height of the metering portion is reduced. The reduced wall thickness is preferably provided only in the region of the joints, and thus only a few areas with a small wall thickness are formed, which can also be kept small in the metering portion with regard to the penetration of oxygen, for example, by diffusion through the container wall. This is advantageous since the stability behavior of oxygen-sensitive products in particular would be reduced by reducing the wall thickness over a large area. Since the metering portion is arranged between the casing portion and the bottom portion, a label can be attached to the casing portion in the usual way.

Preferred embodiments are apparent from the other claims.

According to a preferred embodiment, the bottom portion has a concentrically arranged curvature in the direction towards the metering portion. During use, this curvature of the bottom portion has the effect that the thumb of a user, which is often placed on the bottom to compress the metering portion, automatically comes to rest in the region of the middle of the bottom portion, resulting in uniform compression of the metering portion around the circumference of the substantially rotationally symmetrical storage body of the container.

According to a preferred embodiment, the entire metering portion has a smaller wall thickness than the first wall thickness and preferably the entire metering portion has a wall thickness which is reduced by 25% to 60% with respect to the first wall thickness. If the entire metering portion has a smaller wall thickness than the first wall thickness, a part of the movement of the folding bellows can be effected by the joints, another part of the movement of the folding bellows can also be effected by bending the wall portions of the folding bellows between the joints. This reduces the mechanical stress on the joints.

Preferably, the metering portion has a single fold comprising a first joint on the casing portion, a second joint on the bottom portion, a first annular joint leg which extends radially inward from the first joint and at a first angle relative to the radial direction, a second annular joint leg which extends radially inward from the second joint and at a second angle relative to the radial direction, and a third joint which connects the first joint leg with the second joint leg.

In this regard, it is preferred that the first joint leg and the second joint leg have a substantially linear extension in a sectional plane perpendicular to the longitudinal axis.

In this manner, the metering portion is formed similar to a disk spring which can be elastically compressed in the direction of the longitudinal axis when pressure is applied.

According to a preferred embodiment, the first, second and third joints as well as the second joint leg have a smaller wall thickness than the first wall thickness, and the first joint leg has a greater wall thickness than the second joint leg.

In this manner, when pressure is applied to the bottom portion, the movement of the metering portion is substantially effected in the region of the second annular joint leg close to the bottom portion.

According to a preferred embodiment, the first angle is smaller than the second angle.

This measure also serves to allow the deformation of the folding bellows during compression of the metering portion to be stronger in the region of the second joint adjacent to the bottom portion than in the region of the first joint section adjacent to the casing portion.

In this regard, it is preferred that the second angle is more than twice as large as the first angle, and preferably the second angle is between 30° and 40°.

According to a preferred embodiment of the invention, the second joint is located at a smaller distance from the longitudinal axis than the first joint. This measure also results in that, upon compression, the half of the folding bellows facing the bottom portion performs a stronger movement between the third joint and the second joint than the upper half of the folding bellows, as a result of which it is primarily the metering portion in the region of the second joint that moves upon compression.

Preferably, the ratio between the outer diameter of the bottom portion and the inner diameter of the container in the region of the third joint corresponds approximately to the ratio 2:1.

In this manner, in the region of the metering portion a sufficiently great folding is created in the region of the folding bellows.

According to a preferred embodiment of the invention, the container further comprises an outer thread on the container neck portion. This outer thread serves to screw on a closure cap, by means of which a dispensing opening for dispensing a sterile fluid, which is located in the container neck portion, can be closed.

Furthermore, it is preferred that the container has a shoulder portion between the casing portion and the container neck portion.

Such a shoulder portion can increase ease of use by allowing a user of a small-sized container commonly used to store and dispense eye drops to hold the shoulder portion with the thumb of one hand and a finger of that hand on the outside of the bottom portion. In the case of small-sized containers, such as containers with a content of 5 ml, for example, the container can thus be held comfortably and the pressure exerted on the container bottom when actuating the metering portion can be absorbed, as it were, by a counter bearing while simultaneously holding the shoulder portion.

According to a preferred embodiment of the invention, the container further comprises a circumferential toothing which is provided on the container neck portion and comprises a plurality of substantially tangentially extending ramp surfaces and a plurality of substantially radially extending locking surfaces, each adjoining a ramp surface.

Thus, the toothing on the container neck portion serves to screw a closure cap onto the storage body, which has a conventional tamper-evident ring that is connected with the closure cap via thin-walled bridge portions and can be screwed onto the storage body integrally connected to the closure cap via the toothing after the container has been filled, since the tamper-evident ring can be moved over the tangentially extending ramp surfaces when screwed on for the first time. When opened for the first time, however, the tamper-evident ring detaches from the closure cap since it abuts the locking surfaces and is prevented from following the rotating movement of the closure cap as it is unscrewed, and thus the bridge portions are severed and the tamper-evident ring remains in the region of the toothing, where it gives a clear visual indication to a user that the container has already been opened and the content may no longer be sterile.

The container preferably comprises a corresponding closure cap which can be screwed onto the storage body and, when screwed on, closes the container in an air-tight manner. The measures known in the art serve this purpose, such as the provision of an annular seal on the inside of the top of the closure cap, which, after the closure cap has been screwed on, rests elastically on the outer circumference of a dispensing opening of the container neck portion and in this way seals the container tightly.

### BRIEF DESCRIPTION OF THE DRAWINGS

Hereinafter, the invention will be described, purely by way of example, by means of an embodiment of the container according to the invention. The figures show the following:

FIG. 1 shows a three-dimensional view of the container according to the invention;

FIG. 2 shows a side view of the container according to FIG. 1;

FIG. 3 shows a sectional view of the container according to FIG. 2 along the sectional line A-A; and

FIG. 4 shows a plan view of the container according to FIG. 1.

### WAYS OF IMPLEMENTING THE INVENTION

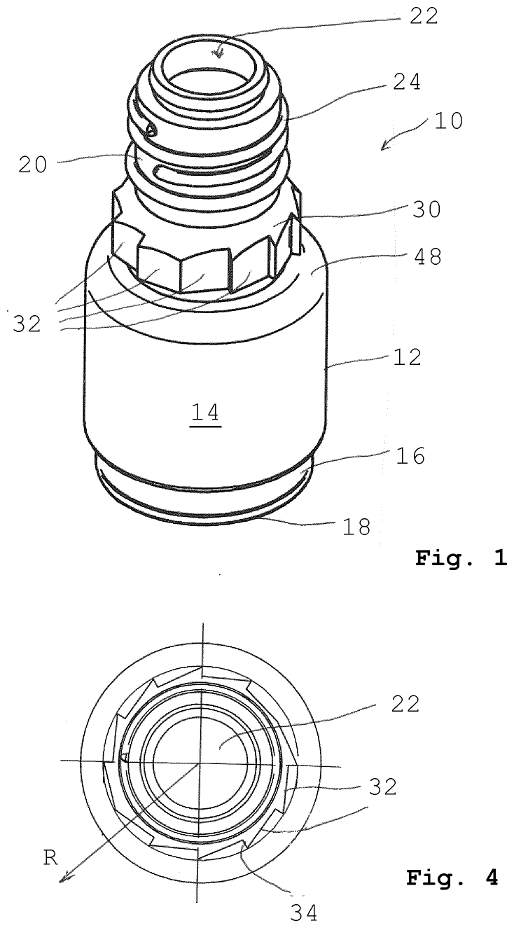

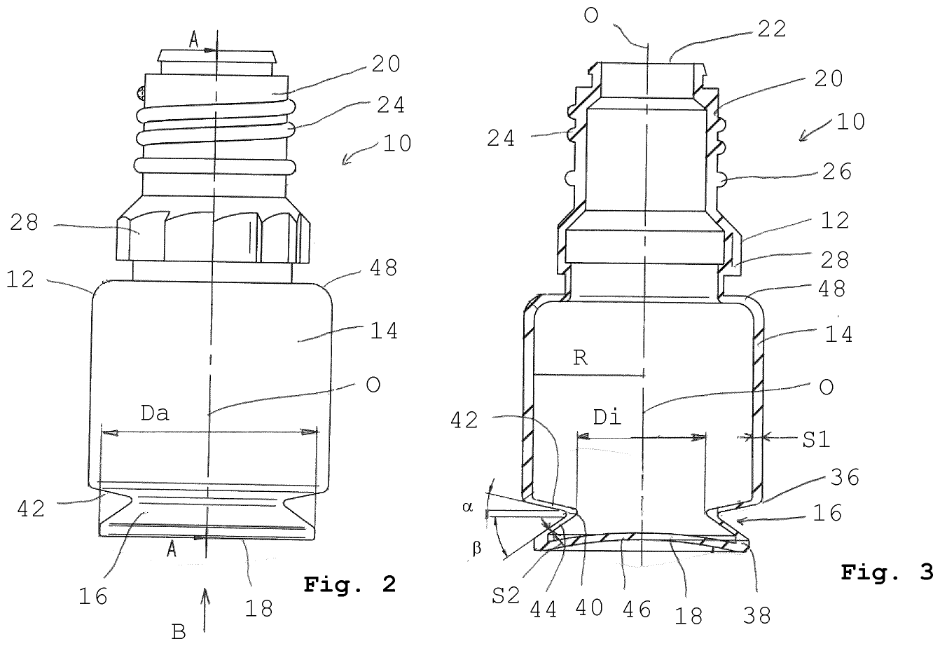

FIGS. 1 to 4 show an embodiment of the container 10 according to the invention for the metered dispensing of a sterile fluid. The container is preferably dimensioned in such a way that a predetermined quantity of sterile fluid is dispensed by actuating the folding bellows, as described further below.

The container comprises a storage body 12 for the sterile fluid as well as a closure cap not illustrated in the figures, which can be screwed onto the storage body. The storage body has a longitudinal axis O and is formed in a substantially rotationally symmetrical manner to the longitudinal axis O. The radial direction R, which is illustrated by way of example in FIG. 4, extends perpendicularly to the longitudinal axis.

The storage body 12 comprises a casing portion 14 which is substantially cylindrical in the present embodiment example, but can also be configured conically, and has a first wall thickness S1. A metering portion 16 integrally adjoins the casing portion 14, and a bottom portion 18 integrally adjoins the metering portion 16. Furthermore, the storage body 12 comprises a container neck portion 20 which has a dispensing opening 22 for dispensing the sterile fluid. An outer thread 24, which serves to screw on a closure cap (not illustrated), is molded onto the container neck portion 20. A circumferential collar 26, which is also illustrated in the figures, serves to be able to hold the storage body in particular during filling with fluid as part of an industrial filling process.

The container according to the present embodiment is additionally provided with a tamper-evident ring 28, which is provided between the outer thread 24 and the casing portion 14 and serves to detach a tamper-evident ring, which is attached to the closure cap in the usual manner via bridging webs, from the closure cap when the container is opened for the first time by unscrewing the closure cap and to make it visible to a user that the container has already been opened. The tamper-evident device 28 can be configured in any way. In the present case, a toothing 30 is provided which comprises a plurality of substantially tangentially oriented ramp portions 32 and locking portions 34 arranged substantially radially between them. As apparent from the plan view according to FIG. 4, a closure cap can be screwed on for the first time in the clockwise direction together with the tamper-evident ring, since the latter is moved along with the closure cap via the ramp portions 32 in accordance with the action of a ratchet, whereas when the closure cap is opened for the first time, the tamper-evident ring is prevented by the locking portions 34 from moving counterclockwise in FIG. 4, and thus the web-shaped bridge portions between the closure cap and the tamper-evident ring tear and the tamper-evident ring remains over the toothing after the closure cap has been unscrewed, while the closure cap can be unscrewed. However, the tamper-evident device used in the illustrated embodiment can be replaced in the same way within the scope of the invention by another tamper-evident device known in the art.

The dimension of the dispensing opening 22 and in particular its diameter is preferably adapted to the formed droplet size of sterile fluid and can have an opening diameter of between 8.1 mm and 8.3 mm for water-based fluids.

The metering portion 16 arranged between the casing portion 14 and the bottom portion 18 is located at a position where it does not collide with the attachment of a label on the casing portion 14 and is thus clearly recognizable by a user. The metering portion 16 is configured as a folding bellows and can have one or several foldings. In the present embodiment, a single folding is provided. For this purpose, the metering portion 16 has a first joint 36, a second joint 38 and a third joint 40. The first joint 36 represents the transition between the casing portion 14 and the metering portion 16. The second joint 38 represents the transition between the bottom portion 18 and the metering portion 16. The third joint 40 is provided inside the folding.

Moreover, a first joint leg 42 is provided which connects the first joint 36 with the third joint 40. A second joint leg 44 connects the third joint 40 with the second joint 38. In the present embodiment example, the joint legs 42 and 44 are configured annularly and have a substantially linear extension in the sectional plane, as illustrated in FIG. 3, perpendicular to the longitudinal axis (O).

The wall thickness (S2) of the metering portion is reduced by 25% to 60% with respect to the first wall thickness (S1). However, it is also possible to configure the first joint leg 42 and the second joint leg 44 with the same wall thickness SI as the casing portion and to provide an accordingly reduced wall thickness merely in the region of the joints 36, 38, 40 in order to enable an increased deformation of the metering portion 16 in these regions.

As also apparent from FIG. 3, angles of different sizes are formed in which, on the one hand, the first joint leg 42 extends relative to the radial direction R and in which, on the other hand, the second joint leg 44 extends relative to the radial direction R. In the specific embodiment example, the angle β between the second joint leg 44 and the radial direction is more than twice as large as the angle α between the first joint leg 42 and the radial direction. In the specific example, the angle a could be between 10° and 15°, whereas the angle β could be between 30° and 40°. The different angles serve to ensure that, when the metering portion 16 is actuated by external pressure on the bottom portion, as described further below, the movement takes place primarily in the region of the second joint 38 and the second joint leg 44. In the same way, however, according to an alternative embodiment, it is also possible to modify the configuration according to FIG. 3 in such a way that actuation takes place substantially in a uniform manner via the first joint leg and the second joint leg, or actuation takes place substantially only via the first joint leg.

Furthermore, it has turned out to be advantageous if the inner diameter Di of the container in the region of the third joint 40 is selected so that it is approximately half as large as the outer diameter Da of the bottom portion 18 in the region of the second joint 38.

Furthermore, it is advantageous if the second joint 38 is located at a smaller radial distance from the longitudinal axis O than the first joint 36 between the casing portion 14 and the metering portion 16. As a result, the radius of the bottom portion 18 is smaller than the radius of the casing portion 14. This geometry, together with the ratio between the dimensions Da:Di, has turned out to be particularly advantageous in terms of the best possible force transmission and force application. The force is applied via the bottom 18 in the direction of the arrow B. Due to the cylindrically symmetrical shape of the bottom 18 and a curvature 46 of the bottom in the direction towards the inner volume of the container, the force is transmitted substantially concentrically. The curvature 46 is advantageous since the user automatically presses in the direction towards the lowest point of the curvature 46 for actuation, and therefore the concentricity of the force application is improved. However, the lever arm is also decisive for the amount of force required. A lever arm that is too small stiffens the system and causes a higher actuating force. However, if the lever length is chosen to be too long, the metering portion becomes too soft depending on the chosen wall thicknesses and not sufficient force is applied to the metering portion configured in the form of a disk spring to actuate it. This would result in that the excess pressure required to form a drop would be too low and no drop would escape from the dispensing opening 22 which serves as a drop hole. Therefore, the chosen lever arm with a ratio of 2:1 between the outer diameter Da of the bottom and the inner diameter Di of the fold has proven to be very suitable for achieving an optimum application of force to form a drop.

However, according to an alternative configuration, the radius of the bottom portion can also correspond to the radius of the casing portion.

The bottom portion 18 itself also has a certain flexibility so that, if sufficient pressure is applied to the bottom, a drop can also be released if only the bottom of the bottle is compressed.

Due to the configuration of the metering portion 16, the container according to the invention can also be made of stiffer plastics materials such as HDPE and PP, which are sometimes used for reasons of resistance to sterile fluid.

It is decisive that at least the joints have a reduced wall thickness for the spring function. It can even be advantageous to provide only the joints with a smaller wall thickness since this reduces the diffusion of oxygen through the outer wall of the container and thus results in better stability behavior for oxygen-sensitive products. Due to the shape, it is intuitively apparent to a user how the container is to be actuated. If a user presses on the middle of the bottom portion 18 in order to compress the metering portion 16, it has turned out to be advantageous to provide a shoulder portion 48 between the casing portion 14 and the container neck portion 20, which acts as a counter bearing, as it were, to facilitate the actuation of the application of force to the bottom portion.

The storage body 12 of the container according to the invention can be made integrally of plastics material, and thus the container is well suited for mass production. By choosing the wall thicknesses in the metering portion, choosing the angles between the first joint leg with respect to the radial direction, the second joint leg to the radial direction and the ratio between the outer diameter Da of the bottom and the inner diameter Di of the fold, an actuating force that is always approximately the same for the user can be set, which is independent of the container volume, the other wall thickness of the container, but also of the chosen plastics material.

1. A container for the metered dispensing of a sterile fluid, the container comprising:
a substantially rotationally symmetrical storage body (12) made of plastics material, having a longitudinal axis (O) and a radial direction (R) extending perpendicularly to the longitudinal axis, wherein the storage body (12) comprises:
a substantially cylindrical or conical casing portion (14) having a first wall thickness (S1),
a metering portion (16),
a bottom portion (18), and
a container neck portion (20); characterized in that:
the metering portion (16) is arranged between the casing portion (14) and the bottom portion (18), and
is configured as a folding bellows having at least one radially inward fold having joints (36, 38, 40); wherein
the wall thickness (S2) of the metering portion (16) is reduced, at least in the region of the joints (36, 38, 40), by 25% to 60% with respect to the first wall thickness S1.

2. The container according to claim 1,
characterized in that
the bottom portion (18) has a concentrically arranged curvature (46) in the direction towards the metering portion (16).

3. Container The container according to claim 1,
characterized in that
the entire metering portion (16) has a smaller wall thickness (S2) than the first wall thickness (S1).

4. The container according to claim 1,
characterized in that
the metering portion (16) has a single fold comprising a first joint (36) on the casing portion (14),
a second joint (38) on the bottom portion (18),
a first annular joint leg (42) which extends radially inward from the first joint (36) and at a first angle (a) relative to the radial direction (R),
a second annular joint leg (44) which extends radially inward from the second joint (38) and at a second angle (B) relative to the radial direction (R), and
a third joint (40) which connects the first joint leg (42) with the second joint leg (44).

5. The container according to claim 4,
characterized in that
the first, second and third joints (36, 38, 40) as well as the second joint leg (44), have a smaller wall thickness than the first wall thickness (S1), and the first joint leg (42) has a greater wall thickness than the second joint leg (44).

6. The container according to claim 4,
characterized in that
the first joint leg (42) and the second joint leg (44) have a substantially linear extension in a sectional plane perpendicular to the longitudinal axis (O).

7. The container according to claim 6,
characterized in that
the first angle (α) is smaller than the second angle (β).

8. The container according to claim 7,
characterized in that
the second angle (β) is more than twice as large as the first angle (α).

9. The container according to claim 4,
characterized in that
the second joint (38) is located at a smaller radial distance from the longitudinal axis (O) than the first joint (36).

10. The container according to claim 4,
characterized in that
the ratio between the outer diameter (Da) of the bottom portion (18) and the inner diameter (Di) of the container in the region of the third joint (40) corresponds approximately to the ratio 2:1.

11. The container according to claim 1, further comprising an outer thread (24) on the container neck portion (20).

12. The container according to claim 1, further comprising a shoulder portion (48) between the casing portion (14) and the container neck portion (20).

13. The container according to claim 1, further comprising a circumferential toothing (30) which is provided on the container neck portion (20) and comprises a plurality of substantially tangentially extending ramp surfaces (32) and a plurality of substantially radially extending locking surfaces (34), each adjoining a ramp surface (32).

14. The container according to claim 1, further comprising a corresponding closure cap which can be screwed onto the storage body (12) and, when screwed on, closes the container tightly.

15. The container according to claim 3, wherein the entire metering portion (16) has a wall thickness which is reduced by 25% to 60% with respect to the first wall thickness (S1).

16. The container according to claim 8, wherein the second angle (β) is between 30° and 40°.

17. The container according to claim 8, characterized in that the first, second and third joints (36, 38, 40) as well as the second joint leg (44), have a smaller wall thickness than the first wall thickness (S1), and the first joint leg (42) has a greater wall thickness than the second joint leg (44).
